SEQUENCE LISTING (1 sequence)

SEQ ID NO: 1 - DNA, 18 bp, Artificial Sequence
  Eub338 probe for bacterial 16S rRNA
gctgcctcccgtaggagt

(19) United States
(12) Patent Application Publication
(10) Pub. No.: US 2010/0323345 A1
(43) Pub. Date: Dec. 23, 2010

(54) METHODS AND COMPOSITIONS FOR IDENTIFYING CELLS BY COMBINATORIAL FLUORESCENCE IMAGING

(76) Inventor: Gary Borisy, Woods Hole, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/867,356

(22) PCT Filed: Feb. 12, 2009
(86) PCT No.: PCT/US09/33882

§ 371 Date: Aug. 12, 2010

Related U.S. Application Data
(60) Provisional application No. 61/065,518, filed on Feb. 13, 2008.

(51) Int. Cl. C12Q 1/68; C12Q 1/70; C07H 21/04

(52) U.S. Cl. 435/5; 435/6; 536/24.3

(57) ABSTRACT

A method of identifying the classification of cells in situ involves labeling the cells with a set of nucleic acid probes and performing combinational fluorescence microscopic imaging. The set of probes contains groups of probes that bind to a taxon-specific or function-specific nucleotide sequence. Each probe of a group of probes is labeled with a distinct fluorescent label, and each group corresponds to a unique combination of labels, which can be detected across the image and serves to identify cells according to a unique taxonomic or functional classification. The combinational labeling and spectral imaging approach expands the number of different classifications that can be identified simultaneously in a single image of a collection of cells. The methods and probe sets of the invention can be used to rapidly identify microbes, study their ecological relationships, screen for novel antibiotics, and identify pathogens.

### CROSS REFERENCE TO RELATED APPLICATIONS

This application claims the priority of U.S. Provisional Application No. 61/065,518 filed Feb. 13, 2008 entitled, METHODS AND COMPOSITIONS FOR IDENTIFYING CELLS BY COMBINATORIAL FLUORESCENCE IMAGING, the whole of which is hereby incorporated by reference.

### FIELD OF THE INVENTION

The invention is related to the identification and characterization of cells such as microorganisms. In particular, the invention is related to methods and compositions for rapidly and simultaneously identifying or characterizing individual cells and populations of microorganisms in a sample using combinatorial fluorescence imaging microscopy.

### BACKGROUND OF THE INVENTION

Identification of microbes such as bacteria has been accomplished using hybridization of fluorescently-labeled oligonucleotide probes, for example by fluorescent in situ hybridization (FISH). Fluorescence and other spectroscopic labeling methods are limited by the number of spectra that can be distinguished by the imaging system. Probe sets labeled with as many as eight distinct fluorophores have been developed for simultaneous hybridization against 16S small subunit RNA and other targets. See U.S. Pat. No. 6,738,502. Libraries of probes have been used with spectral deconvolution software to identify complex mixtures of cells by spectral sorting. The use of distinct labels for each probe limits the number of probes that can be detected simultaneously to the small number of labels that can be sorted out by spectral deconvolution. However, there remains a need to simultaneously detect up to hundreds of distinct organisms in order to analyze populations of cells.

### BRIEF DESCRIPTION OF THE DRAWINGS

FIG. 1 shows the fluorescence emission spectra of eight different fluorophores, as indicated, conjugated to Eub338, a universal eubacterial nucleic acid probe. The probe was added to E. coli cells, and the spectra recorded using a Nikon C1si LSCM imaging fluorescence microscope.

FIGS. 2A-2F show a flow diagram of the image processing steps involved in identifying 28 differently labeled batches of E. coli cells, each binary labeled with two of the eight different conjugated Eub338 probes of FIG. 1.

FIGS. 3A-3D show the results of the labeling of E. coli cells as outlined in FIG. 2. FIG. 3A shows an image of a preparation of E. coli cells containing a mixture of 28 differently labeled batches of cells, each binary labeled by FISH with two of the eight different conjugated Eub338 probes of FIG. 1. The image contains approximately 3,000 cells and was acquired using a 20× 0.75 N.A. objective and a Nikon C1si LSCM fluorescence microscope. The scale bar represents 50 μm. FIG. 3B shows a magnified view of the area marked with a box on FIG. 3A. Each cell is marked with a two-letter code representing the binary label recorded from that cell. The letter codes represent the dyes listed in FIG. 1. FIG. 3C shows the distribution of labeled E. coli cells in FIG. 3A, using the same labeling scheme used in FIG. 3A. Each wedge represents a unique binary labeling combination, as indicated in the figure. FIG. 3D shows a comparison of the relative proportion of the different binary-labeled E. coli cells of FIG. 3C (open bars) with cell concentrations calculated from hemacytometer counts (shaded bars) of each binary-labeled batch of cells added to the mixture.

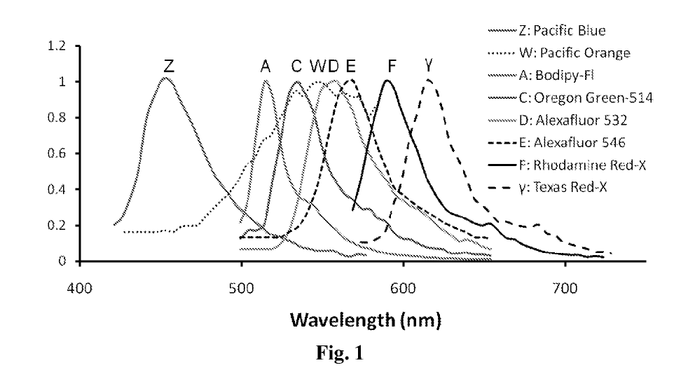

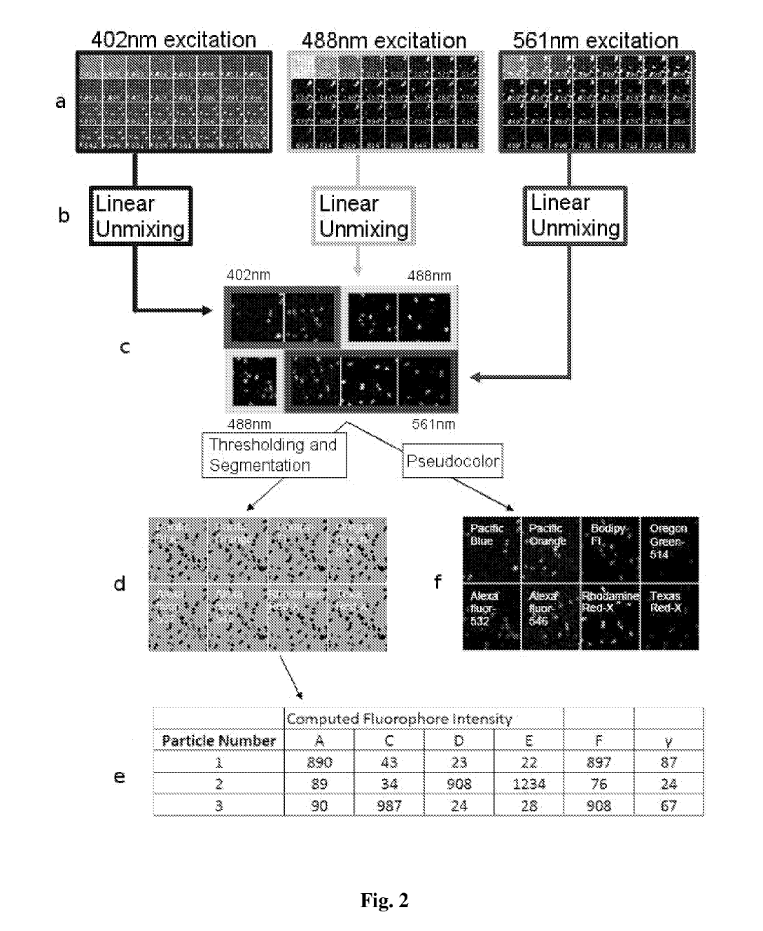

### SUMMARY OF THE INVENTION

The invention provides methods and compositions for the identification and taxonomic or functional classification of cells, and especially microbes. The invention also permits characterization of important features of the genome or expression of cells (e.g., antibiotic resistance genes or genes for particular metabolic functions). The methods and compositions are based on a combinatorial labeling strategy that permits a large number of classifications (e.g., species) to be identified simultaneously in a high throughput format without the need for nucleic acid isolation or amplification and without the need for culturing. Microorganisms are identified individually by fluorescence imaging, and their ecological relationships as well as their role in pathology can be investigated. The methods and compositions of the invention also can be used to develop new antimicrobial agents, to detect pathogens, to study the effects of various agents on microbial population dynamics, and to analyze gene expression in cells and tissues.

One aspect of the invention is a method of identifying a taxonomic or functional classification of microbes, such as bacteria, archaea, fungi, algae, or microscopic eukaryotes by fluorescent in situ hybridization. The method includes the steps of: (a) providing a sample containing one or more cells, such as microbes; (b) incubating the sample with a set of fluorescently labeled nucleic acid probes; (c) imaging the sample using a fluorescence microscope; and (d) analyzing the image to identify a taxonomic or functional classification of the microbe. The set of labeled probes comprises one or more groups, each group including a first nucleic acid probe and a second nucleic acid probe, with each group of probes bound to a unique combination of fluorescent labels that represents a single taxonomic or functional classification of cell. The first probe of each pair is bound to a first fluorescent label, and the second probe of the pair is bound to a second fluorescent label. All or a portion of the sequence of each probe is complementary to an identifier sequence present in some or all cells of the unique taxonomic or functional classification being identified, such that the probe hybridizes to those cells when FISH is performed. The first probe of each group can be identical or nearly identical, or can be non-identical in sequence to the second probe of the group. In a preferred embodiment, the identifier sequence is a 16S ribosomal RNA sequence. The taxonomic classification identified can be a classification such as a domain, phylum, class, order, family, genus, species, subspecies, strain, or clade. Alternatively, a functional classification can be identified through either the presence or expression of one or more genes in the cells of the sample. The sample containing cells or microbes for identification or characterization can be obtained from a source such as seawater, surface water, ground water, drinking water, tap water, air, a surface wipe sample, an industrial product or effluent, a food or beverage, a probiotic or synbiotic preparation, a fermentation broth or cell culture medium, a biofilm, a medical implant, or a patient sample. The patient sample can be a material suspected of containing a pathogenic microbe, and the method can be used for diagnosing a disease or medical condition. In certain embodiments of the method, at least one group of probes in the set contains a third nucleic acid probe, which is coupled to a third fluorescent label. The first, second, and third labels of the group form an identifiably unique combination of labels that represent and identify a particular taxonomic or functional classification of cells.

Another aspect of the invention is a method of determining a taxonomic or functional classification distribution for a population of cells such as microbes. Steps (a) through (d) of the method described above are first performed, followed by (e) determining a taxonomic or functional classification distribution for the population. In one embodiment of the method, changes over time in such a distribution are determined by (f) comparing the taxonomic or functional classification distributions obtained at the beginning and end of a time interval to identify a change in the distribution over the time interval.

Yet another aspect of the invention is a method for determining the effect of a chemical, physical, or biological agent on a population of cells such as microbes. The method includes performing steps (a) through (e) above followed by repeating the same steps after contacting the population of microbes with a chemical, physical, or biological agent, and (f) comparing the taxonomic or functional classification distributions to identify a change in the distribution in response to the agent. The chemical agent is a chemical moiety such as an antimicrobial agent, a pharmaceutical agent, a nucleic acid, a nutrient, a food, a beverage, a prebiotic, probiotic or synbiotic preparation, a fermentation broth, a cell culture medium, a water sample, an air sample, a pollutant, or a patient sample. In one embodiment of the method, the agent is an antimicrobial agent; this method can be used to develop new antibiotics. The physical agent is an agent such as heat, cold, an electromagnetic radiation or field, a radioisotope emission, cosmic radiation, or a particle beam.

A further aspect of the invention is a set of fluorescently labeled nucleic acid probes. The set includes one or more groups of probes, each group containing a first nucleic acid probe and a second nucleic acid probe. The first probe of each group can be identical or nearly identical, or can be non-identical in sequence to the second probe of the group. The first probe is bound to a first fluorescent label, and the second probe is bound to a second fluorescent label. The first and second fluorescent labels of each group of probes are a unique combination within the set. Further, all or a portion of the sequence of each probe is complementary to and hybridizes with an identifier sequence present in some or all of the cells of a unique taxonomic classification, such as a species. In certain embodiments of the set of probes, at least one group of probes in the set contains a third nucleic acid probe, which is coupled to a third fluorescent label. The first, second, and third labels of the group form an identifiably unique combination of labels that represent and identify a particular taxonomic or functional classification of cells.

Still another aspect of the invention is a kit that includes one or more sets of fluorescently labeled nucleic acid probes together with packaging materials and instructions for use. Each set of labeled probes includes one or more groups of first and second nucleic acid probes. The first probe of each group can be identical or nearly identical, or can be non-identical in sequence to the second probe of the group. The first probe is bound to a first fluorescent label, and the second probe is bound to a second fluorescent label. The first and second fluorescent labels of each group of probes are a unique combination within the set. Further, each probe hybridizes to an identifier sequence present in some or all cells of a unique taxonomic or functional classification, such as a species.

### DETAILED DESCRIPTION

The present invention utilizes a combinatorial labeling and imaging approach to greatly expand, compared to previous methods, the number of different taxonomic or functional classifications that can be identified simultaneously in a single image of a collection of cells such as microbes. The particular binary and ternary labeling strategies described here permit up to hundreds of distinct cell types, such as species of bacteria, or bacteria possessing one or more functional genes (e.g., for antibiotic resistance or for particular metabolic activities) to be simultaneously identified in a single image. For the first time, an entire naturally occurring population of microbes or other cells can be identified or characterized in a single image.

Previously available alternatives for identifying numerous species in a single sample or population required either serial labeling and multiple images, which is time consuming, or fluorescence-activated cell sorting (FACS), in which spatial relationships between cells are lost. However, the combinatorial imaging approach employs a binary or ternary labeling strategy that improves spectral resolution and increases the number of different probe sequences that can be simultaneously imaged. The increased labeling possibilities permit the rapid identification or characterization of a large number of microbial species or other taxonomic classifications as well as the identification of spatial relationships within naturally occurring communities or tissues over a scale up to 100 μm or even 1 mm. The imaging approach further provides awareness of which cells, if any, do not react with any of the probes in a given set, information that is also not provided by FACS.

### Combinatorial Imaging

The present invention uses sets containing groups of distinctly labeled duplicate or triplicate nucleic acid probes which may be of identical, nearly identical, or non-identical nucleotide sequence; however, whether or not identical, each probe of a set will hybridize selectively with cells of a particular type for purposes of identification or characterization. Each taxonomic classification (taxon) or functional classification to be identified in a sample is specified by a group of usually either two or three nucleic acids possessing a nucleotide sequence or set of sequences that is specific for the taxon. Each nucleic acid probe of a taxon-specific group (e.g., a pair or triplet) is labeled with a uniquely distinguishable label, such as a fluorophore. A set of probes for the identification of a selected group of taxa contains a probe pair or triplet for each taxon in the group. While each group of probes will have both a nucleotide sequence (or in some embodiments a plurality of nucleotide sequences) and a corresponding combination of labels that is unique within the probe set, the individual labels are generally not unique within the set, and can appear in more than one combination of labels. Each taxon-specific or function-specific combination of labels in the library is capable of unique and unambiguous identification in a microscope image based upon the combined spectral signature of the selected labels.

In principle there is no upper limit to the number of nucleic acid probes within a group. However, the combinatorial labeling approach permits a great diversity of cell classifications to be identified using only a small number of probes in a group, depending in part on the number of fluorescent labels that can be distinguished in a given sample, and thus can be used together in a set (see below). Using either two or three probes per group, together with a small number of fluorescent labels, such as 8 to 15, hundreds of taxa or functional classes can be detected in a sample from a single analysis. The probes of any given group will all hybridize with the same target, a selected taxonomic or functional classification of cells, by hybridizing to one or more identifier sequence(s) in such cells. Each individual probe of a group will hybridize to a single identifier sequence. However, the probes of a group can each hybridize either to the same identifier sequence (in which case the probes of the group differ only by their attached labels), or to different identifier sequences that are nevertheless all characteristic for the same target cell classification (in which case the probes of the group differ both by their sequences and by their attached labels).

A set of probes can contain as many groups as needed to identify as many different taxa or functional classifications as are found, or expected to be found, in a given sample.

For example, a set can contain only one group, or can contain 2 or more groups, 2-10 groups, not more than 15, not more than 28, not more than 45, or not more than 105 groups, depending on the number of fluorescent labels that can be distinguished in the presence of one another by the fluorescence microscopy system available. In the general case, a set of probes can contain not more than n(n−1)/2 groups when binary labeling is used, and not more than n(n−1)(n−2)/6 if ternary labeling is used, where n is the number of labels in the set. Thus, for binary labeling, a set of probes can contain not more than 15 groups when 6 labels are used, not more than 21 groups when 7 labels are used, not more than 28 groups when 8 labels are used, and so forth.

Fluorescence imaging has been used extensively to identify individual molecular components of organelles, cells and tissues (Lichtman et al., 2005). Most studies have used a single fluorescent reporter molecule, or a small number (usually not more than 2-3), of such reporters in a single image. This is due to the limitations of using different band pass filters to specify the required portion of both the excitation and emission spectra of each fluorophore. Recent advances in spectral imaging have overcome the limitations of band pass filters and have allowed greater use of combinations of fluorescent probes (Hiraoka et al., 2002; Garini et al., 2006). Combinations of a small number of distinct probes can be used to extend the number of uniquely identifiable structures, nucleotide sequences, or cells. Generally, the number of distinct combinations of n fluorophores is 2ⁿ−1. For example, using five distinct fluorophores, 31 distinct targets can be detected. This approach has been used to karyotype human cells, having up to 24 different chromosomes (22 autosomes plus X and Y) (Schrock et al., 1996). Using multichannel fluorescence detection, up to 11 different fluorophores have been used in a single probe for FACS (DeRosa et al., 2001). Using existing spectral imaging technology, the simultaneous detection of at least 15 different fluorophores spread over the visible spectrum is currently possible, and more are possible by using the infrared region. Combining n different labels in binary combinations results in n(n−1)/2 unique combinations. Combining n different labels in ternary combinations results in n(n−1)(n−2)/3*2 unique combinations. Thus, 15 fluorophores can be combined into 105 binary probes or 455 ternary probes, each of which can be used for taxon-specific detection and visualization. Using such ternary probes, nearly all of the estimated 500 species of microbes that inhabit the human mouth (Paster et al., 2001) could be detected, or a substantial fraction of the 1000 species that inhabit the human gut (Hooper et al., 2001).

### Cell Types Identified

The probe sets and methods of the present invention can be used to identify a wide variety of taxonomic classifications of microbial species as well as to identify genetic variations within a tissue from an animal or plant. In principle, any biological material that contains genetic variations and can be examined under a microscope can be used as a sample for cell identification according to the invention. Probes can be designed based on sequences that are partially conserved across species, such as rRNA sequences, or based on taxon-specific genes, or based on particular structure- or function-related genes found in many species such as genes for particular enzymes or metabolic pathways. Probes can also be based on mRNA sequences in order to analyze gene expression across a tissue or a population of cells. In a preferred embodiment, the probes are based on rRNA sequences. Taxonomic classifications that can be analyzed include not only species, but also phyla and other classifications. For example, a taxon selected from domain, phylum, class, order, family, genus, species, subspecies, strain, and clade can be identified using methods or compositions according to the invention.

The identification of unknown cells by hybridization to rRNA can be performed for bacteria and archaea as well as other cell types. Eucarya such as ascomycetes, algae, protists and other cells can be identified by amplifying their small subunit (e.g., 18S) or large subunit (e.g., 23S) rRNA, sequencing the RNA genes, designing probes based on the sequences, and hybridizing the probes to the cells (Medlin et al., 1988; Lim et al., 1993). Mammalian cell 18S-28S rRNA also can be probed with fluorescent nucleic acids (Labidi et al., 1990), and fluorescent probes to the 16S rRNA of human mitochondria have been hybridized in situ to skeletal muscle tissue to identify cells with mitochondrial disorders (Hilton et al., 1994).

Identification of cells is an integral part of biological taxonomy, and it has numerous additional uses in medicine, environmental studies, and public safety. Medical uses include confirming bacterial serotypes for epidemiological studies (Birnbaum et al., 1994) and monitoring of nosocomial infection (Andersen, 1995). Environmental uses include analysis of water, soil and air, as well as bioremediation monitoring (Schrenk et al., 1998) and studies of population ecology and bacterial phylogenetics (Pace et al., 1986; Ward et al., 1992; Amann et al., 1995). In biotechnology, taxonomic identification can be used for biodiversity screening, bioprocess monitoring and genomic analysis (Amann et al., 1992; Hoheisel, 1997; Head et al., 1998).

Traditionally, identification of microbes has relied on their growth in culture, despite the knowledge that most of them are not cultivable by standard methods (Amann et al., 1995; Pace, 1997; Head et al., 1998; Hugenholtz et al., 1998b). Recently, molecular methods have been developed to enable the identification of microorganisms without the need to isolate or culture them. One class of methodology takes advantage of the conserved nature of protein synthesis in all organisms. With about 200,000 partial or complete sequences now available for comparison, the small subunit ribosomal RNA (rRNA, which contains the 16S rRNA in bacteria and archaea and the 18S rRNA in eucarya) is currently the preferred molecule for identifying organisms at the species level. Other suitable rRNA targets include the 5S or 23S rRNA in bacteria and archaea and the 5S, 5.8S and 28S rRNA in eucarya. Molecular strategies based on PCR, cloning, sequencing, and probing have enabled biologists to examine the total microbial community in a sample without any a priori knowledge of the species present in the mixture (Amann et al., 1995).

### Probe Design

Probes for use in the methods of the inventions are nucleic acids or nucleic acid analogs or derivatives capable of selectively hybridizing in situ to a target sequence (identifier sequence) within a cell having a taxonomic classification that is to be identified in a sample. For example, peptide nucleic acids can be used as probes. Generally, probes for use in the invention are nucleic acids or analogs or derivatives of nucleic acids of approximately 15-30 bases in length; preferably they are about 18-22 bases in length, or about 18 bases in length. Probes can also be longer if required, e.g., where the target sequence is found in low abundance in the cell class to be detected. In such cases, it may be advantageous to hybridize a plurality of identical probe molecules to a single target molecule, so as to increase the signal intensity. Alternatively, probes can be prepared having two or more label moieties attached to each probe molecule, so as to increase the signal intensity. Thus, a probe can be up to 100 kilobases in length, and may contain anywhere from 1 to 100 or more, or even 1000 or more molecules of attached fluorophore molecule per probe molecule. In some cases the probe can be fragmented into pieces 20-500 bases in length, preferably 50-100 bases in length, for better penetration of the probe into the target cells. If a fragmented probe is used, then each fragment preferably bears at least one label moiety, such as a fluorophore. All or a substantial portion of the probe nucleotide sequence will hybridize to a target sequence found in target cells. In addition to the hybridizing sequence, a nucleic acid probe according to the invention can optionally include one or more linker portions or additional segments as desired or as needed to attach one or more labels to the hybridizing sequence or to modify the tertiary structure, solubility, or cell permeability of the probe.

Although rRNA-based identification is accurate to the level of species, its versatility makes it valuable for high-throughput screening and identification of microorganisms. The information gained from 16S/18S rRNA sequence comparisons can be used to deduce detailed phylogenetic relationships based on evolution. The highly conserved portions of 16S/18S rRNA are ideal for designing primers that will amplify 16S/18S rRNA genes from all three domains of life (Bacteria, Archaea, and Eucarya). At the other extreme, primers can be designed to highly variable regions of 16S/18S rRNA and thus amplify only a particular species or genus in a mixture of microorganisms Likewise, fluorescent DNA hybridization probes based on 16S/18S sequencing information can be constructed to identify organisms in a large group such as a phylum, or in a smaller group such as a genus, depending on whether the probe sequence is complementary to a conserved or variable region of the 16S/18S rRNA, respectively. Ribosomal RNA is a particularly convenient and attractive hybridization target for quantitative microscopy because of the number of copies per cell (thousands to tens of thousands for an exponentially growing bacterial cell). Nucleic acid sequences that are present only once or a small number of times per cell may also be used as targets, in which case the nucleic acid target must be longer and/or the number of fluorophores per probe must be increased in order that enough fluorescently-tagged probe molecules may hybridize to the target or that enough signal is generated by a target cell. PCR has been used to amplify the 16S-rDNA genes from microorganisms isolated from diverse environments as well as from clinical sources (Hugenholtz et al., 1998b; Relman, 1998). Unknown bacteria continue to be identified at the level of new phyla. Many of these new phyla have no cultured representatives, yet PCR analysis indicates that they are abundant in the environment. These organisms are likely to become a rich source of new antibiotics, enzymes, and other bioactive compounds for medicine and biotechnology. However, to examine the diversity of microbial populations containing uncultured species, a partial or full length 16S rRNA sequence should be available for probe design.

Probes for use with the invention can be made using polymers other than DNA. Such polymers include RNA and nucleic acid analogues such as peptide-nucleic acids, phosphorothioates, and morpholinos. Probes can be labeled either covalently or non-covalently (e.g., by hybridization) with fluorophores or other spectroscopically identifiable labels to enable in situ hybridization and identification by fluorescence or other spectroscopic imaging microscopy (Amann et al., 1990). The probes can also contain fluorophores designed to function by fluorescence resonance energy transfer (FRET) or to serve as molecular beacons, i.e., a pair of labels that are quenched in the unbound state but fluoresce when bound to their target sequence.

### Sample Preparation

A sample for analysis can be a small amount of a material, generally at least 1 μL in volume, obtained from an environmental source, from an artificial, industrial, or laboratory source, or from an animal or plant source. A sample can be obtained by direct collection of a liquid or solid material, or can be obtained by using a wipe or swab on a surface, or concentrated from air or liquid by filtration. Samples for analysis include, for example, blood, plasma, serum, sputum, urine, feces, gastric fluid, mucous, vaginal fluid, semen, tears, wound fluid, cerebrospinal fluid, biopsy material, cells, or a tissue sample from an animal, or seawater, brackish water, surface freshwater, ground water, drinking water, tap water, air, a surface wipe sample, the surface itself of a household, medical, industrial, environmental, or laboratory object, an industrial product or effluent, food, beverage, fermentation broth, cell culture medium, a biofilm, or a medical implant.

In a preferred embodiment, sample preparation is similar to sample preparation for FRET analysis. A portion of the sample is placed on a microscope slide or into a chamber suitable for preservation of the cells for analysis. For example, the sample chamber can include conditions of temperature, pressure, metabolites, gases, growth factors, culture medium, electrolytes, or other components as desired to establish or maintain the viability, metabolic state, or physiological state of the cells to be analyzed. If desired, one or more detergents or other permeabilizing agents can be added to improve access of the probes to the interior of the cells. A further option is to add one or more fixatives to assure that biomolecules are retained for detection by the probes. After addition of the probes at 0.5 to 4 micromolar (preferably 1 to 2 micromolar) each, a suitable incubation period is allowed for uptake of the probes (between 15 minutes and 16 hours, preferably 2 hours) followed by one or more washes in probe-free hybridization buffer or a similar solution to remove unbound probe. Conditions can be chosen to promote hybridization of probes with identifier sequences in target cells. See, e.g., Perry-O'Keefe et al., (2001)). For example, the incubation can be carried out at elevated temperature (e.g., 45-70° C., depending on the melting temperatures of the probes or of the identifier sequences. In certain embodiments, probe sequences within a set are chosen such that their melting temperatures are in the same range, e.g., within 5, 7, 8, 10, or 15° C. of one another. The incubation temperature can be chosen to be lower than the melting temperature of the probes in the set and lower than the melting temperature of the identifier sequences in the target cells. Alternatively, the incubation temperature can be at about the melting temperature of the probes in the set, or slightly higher.

### Imaging and Analysis

Available imaging techniques can be utilized to obtain a color image of the sample that permits each unique combination of labels in the set to be distinguished from all other combinations of labels in the set. For example, if the labels are fluorescent moieties, a suitable excitation light source is applied to the sample which is capable of providing excitation at the required wavelengths for all labels in the set, and light emitted from the sample is detected at the appropriate wavelengths so as to permit distinguishing each separate label combination in the set from all other label combinations in the set. The use of a multi-anode photomultiplier tube spectral imager provides sufficient spectral resolution at each point in the image for detecting unique label combinations. In a preferred embodiment, imaging is performed using a 32-channel spectral imaging confocal laser scanning microscope such as a Nikon C1si or a Zeiss LSM 510 meta. An appropriate selection of lasers is used to provide the excitation wavelengths required for the labels in the set, while the spectral imaging system provides sufficient definition of the combined emission spectra of all the dyes in the set to support spectral deconvolution and subsequent identification of each taxon-specific label combination present in each part of the image. Typically, the image is recorded using a multi-anode photomultiplier tube and stored and analyzed using a computer.

Another preferred imaging system involves the placement of the LightForm (LightForm, Inc., Hillsborough, N.J.) PARISS curved prism (see U.S. Pat. No. 5,127,728 and lightforminc.com/PARISSHowTo.html), in the optical path before a CCD camera. This allows full spectral resolution at each point along a line (y-dimension) in the image. By translation of the specimen in the x-dimension, full spectral resolution is obtained at each point in the image in both x- and y-dimensions.

For data acquisition, the same field of view can be imaged sequentially with up to three to four or more excitation sources (e.g., 561 nm laser, 488 nm laser, 402 nm laser). Each of the separate spectral acquisitions is then unmixed using a computer and an unmixing algorithm. Unmixing can be performed, for example, using a linear unmixing algorithm. Linear unmixing algorithms are known in the art; see, e.g., Dickinson et al., 2001. Unmixing can also be performed using the linear unmixing algorithm incorporated into Nikon EZC1 software. The unmixed data then can be exported into an analysis program such as ImageJ, Mathematica, or MatLab, where the relevant pure fluorophore channels are combined into a single multiple-channel image. The number of channels usually will correspond to the total number of different labels used in the set of probes. Each channel of this image displays the computed fluorescence intensity of one of the fluorophores used in the experiment. Cells are segmented from background in each of the channels by selecting pixels whose gray value is above a certain threshold. The threshold is determined using an isodata algorithm, e.g., the algorithm incorporated into ImageJ. See, e.g., Rasband, W. S., ImageJ. U.S. National Institutes of Health, Bethesda, Md., USA, available at rsb.info.nih.gov/ij, 1997-2007). A binary mask (see, e.g., Russ, J. C. The Image Processing Handbook, 2nd ed., CRC Press, Boca Raton, Fla. 1995, pages 414-416) is generated and applied sequentially to each of the channels. Fluorophore intensities under the mask are tabulated and a matrix is generated showing the intensity measurement for each channel in each particle in the field. In an experiment with binary labels, the two channels with the highest intensity are considered to be positive. Quality control is accomplished by comparing the intensities of the third-brightest and second-brightest channels; unless the difference between these intensities is greater than a chosen threshold amount, the cell assignment is considered ambiguous. For example, if the intensity of the third brightest channel for a given cell is within 30% of the second brightest, the cell assignment would be considered ambiguous using a 30% threshold. Data can be analyzed in tabular form. For presentation, the individual channels of a multi-channel image can be pseudo-colored and then merged using the logical operator “OR” to generate a combinatorial image.

### Examples

### Example 1

### Combinatorial Imaging of Bacterial Cells

E. coli cells were hybridized with all 28 binary combinations of eight fluorophore-tagged probes to generate 28 spectral signatures. The probes used were the 18-base oligonucleotide Eub338 (5′ GCTGCCTCCCGTAGGAGT 3′), tagged at the 5′ end with one of the following eight fluorophores: Bodipy-FL (A), Oregon Green 514 (C), Alexa 532 (D), Alexa 546 (E), Rhodamine Red X (F), Texas Red X (gamma), Pacific Orange (W), and Pacific Blue (Z). The 5′ labeled oligonucleotides were procured from Invitrogen Corp. The fluorophores were attached to the oligonucleotides via an amide linkage via a succinimidyl ester of a primary amine. Cells were grown to log phase, harvested, fixed with 2% paraformaldehyde for 1 to 2 hours, washed, and stored in 50% phosphate-buffered saline and 50% ethanol at −20 degrees C. prior to hybridization. Cells were collected by centrifugation and resuspended in a hybridization buffer of 0.9 M NaCl, 20 mM Tris pH 7.4, 0.01% SDS, and 20% formamide. For each batch of cells, two probes were added to the hybridization solution at a concentration of 2 micromolar for each probe. Hybridization was conducted at 46 degrees C. for 2 to 2.5 hours, followed by washing in probe- and SDS-free hybridization buffer, then a final wash in NaCl and Tris only. Aliquots of hybridized, washed cells from all 28 batches were combined in a single tube for spotting onto a microscope slide and imaging; the remaining cells from each batch were counted with a hemacytometer to estimate the number of each type of cell that was put into the mixture.

A Nikon C1si microscope with a 20× 0.75 N.A. dry objective was used to image each field of view sequentially using excitation by a 561 nm laser, a 488 nm laser, and finally a 402 nm laser. Each of these three separate spectral acquisitions was then unmixed using Nikon EZC1 software; the data were then exported into ImageJ, where the eight pure fluorophore channels were combined into a single eight-channel image. Cells were segmented from background in each of the channels using an isodata algorithm. A binary mask was generated and applied sequentially to each of the channels. Fluorophore intensities under the mask were tabulated and a matrix was generated showing the intensity measurement for each channel in each particle in the field.

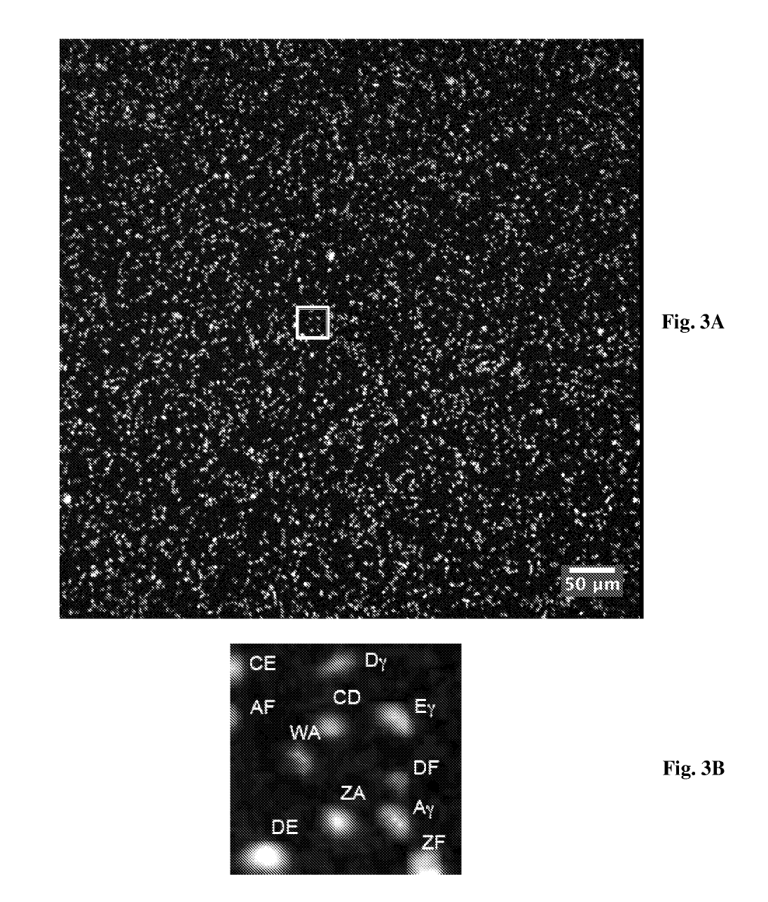

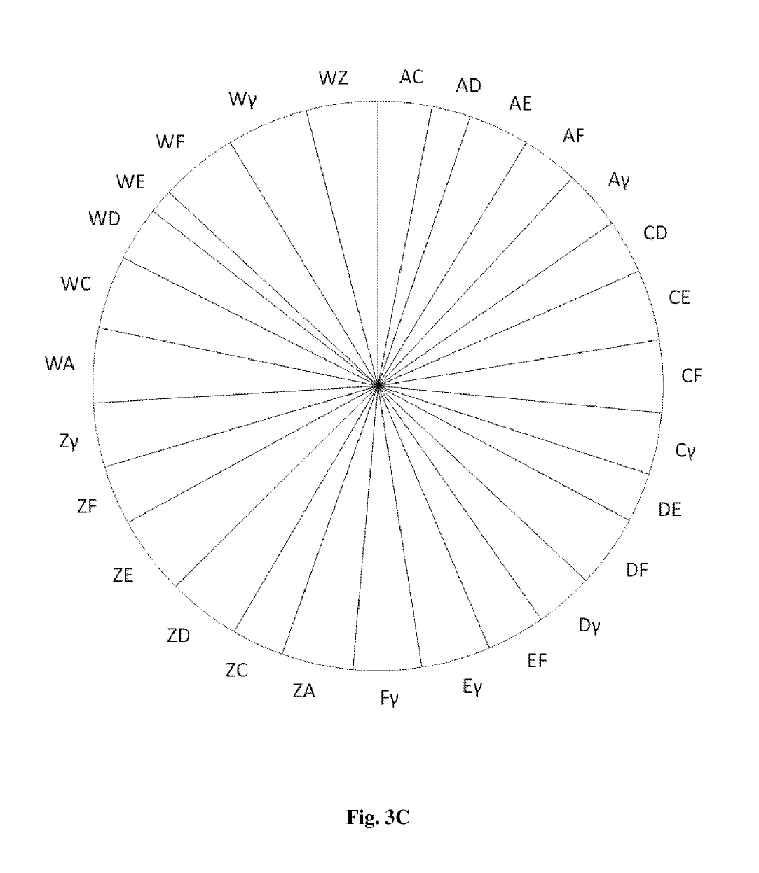

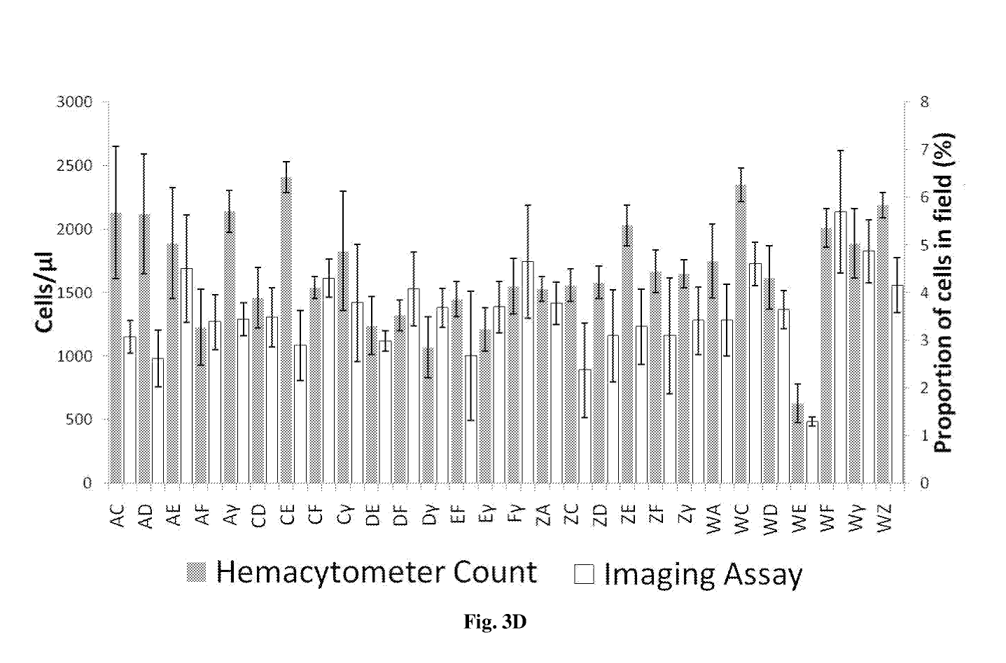

FIG. 3A shows a field with approximately 3000 cells visible. All 28 spectral signatures are readily distinguishable, and the relative proportions of each type of labeled cell in the field of view corresponded well with the proportions of cells input into the mixture (FIGS. 3C, 3D).

### REFERENCES

Amann, R. I., Krumholz, L. & Stahl, D. A. (1990) Fluorescent-oligonucleotide probing of whole cells for determinative, phylogenetic, and environmental studies in microbiology. J. Bacteriol. 172:762-770.
Amann, R. I., Stromley, J., Devereux, R., Key, R. & Stahl, D. A. (1992) Molecular and microscopic identification of sulfate-reducing bacteria in multispecies biofilms. Appl. Environ. Microbiol. 58:614-623.
Amann, R. I., Ludwig, W. & Schleifer, K.-H. (1995) Phylogenetic identification and in situ detection of individual microbial cells without cultivation. Microbiol. Rev. 59:143-169.
Andersen, B. M. (1995) Biochemical profiles and serotypes of nosocomial Enterobacter cloacae strains in Northern Norway: Biochemical identification problems with commercial test systems. Infection 23:339-343.
Birnbaum, D., Herwaldt, L., Low, D. E., Noble, M., Pfaller, M., Sherertz, R. & Chow, A. W. (1994) Efficacy of microbial identification system for epidemiologic typing of coagulase-negative staphylococci. J. Clin. Microbiol. 32:2113-2119.
Dickinson, M. E., Bearman, G., Tille, S., Lansford, R., and Fraser, S. E. (2001) Multi-spectral imaging and linear unmixing add a whole new dimension to laser scanning fluorescence microscopy. BioTechniques 31:1272-1278.
Head, I. M., Saunders, J. R. & Pickup, R. W. (1998) Microbial evolution, diversity, and ecology: A decade of ribosomal RNA analysis of uncultured microorganisms. Microb. Ecol. 35:1-21.
Hilton, D. A., Love, S., Goodwin, T. & Pringle, J. H. (1994) Demonstration of mitochondrial ribosomal RNA in frozen and paraffin-embedded sections of skeletal muscle by in situ hybridization. Neuropathol. Appl. Neurobiol. 20:573-576.

Hoheisel, J. D. (1997) Oligomer-chip technology. Trends Biotechnol. 15:465-469.
Hugenholtz, P., Goebel, B. M. & Pace, N. R. (1998) Impact of culture-independent studies on the emerging phylogenetic view of bacterial diversity. J. Bacteriol. 180:4765-4774.
Labidi, B., Broders, F., Meyer, J. L. & Hernandez-Verdun, D. (1990) Distribution of rDNA and 28S, 18S, and 5S rRNA in micronuclei containing a single chromosome. Biochem. Cell. Biol. 68:957-964.
Lim, E. L., Amaral, L. A., Caron, D. A. & DeLong, E. F. (1993) Application of rRNA-based probes for observing marine nanoplanktonic protists. Appl. Environ. Microbiol. 59:1647-1655.
Medlin, L., Elwood, H. J., Stickel, S. & Sogin, M. L. (1988) The characterization of enzymatically amplified eukaryotic 16S-like rRNA-coding regions. Gene 71:491-499.
Pace, N. R. & Stahl, D. A. (1986). Microbial ecology and evolution: a ribosomal RNA approach. Annu. Rev. Microbiol. 40:337-365.
Pace, N. R., Stahl, D. A., Lane, D. L. & Olsen, G. J. (1986). The analysis of natural microbial populations by rRNA sequences. Adv. Microb. Ecol. 9:1-55.
Perry-O'Keefe, H., Rigby, S., Oliveira, K., Sorensen, D, Stender, H., Coull, J., and Hyldig-Nielsen, J. J. (2001) Identification of indicator microorganisms using a standardized PNA FISH method. J. Microbiol. Meth. 47:281-292.
Relman, D. A. (1998) Detection and identification of previously unrecognized microbial pathogens. Emerg. Infect. Dis. 4:382-389.
Schrenk, M. O., Edwards, K. J., Goodman, R. M., Hamers, R. J. & Banfield, J. F. (1998) Distribution of thiobacillus ferrooxidans and leptospirillum ferrooxidans: implications for generation of acid mine drainage. Science 279:1519-1522.
Ward, D. M., Bateson, M. M., Weller, R. & Ruff-Roberts, A. L. (1992) Ribosomal RNA analysis of microorganisms as they occur in nature. In: Advances in Microbial Ecology (Marshall, K. C., ed.) pp. 219-286, Plenum Press, New York.

1. A method of identifying a taxonomic or functional classification of a cell by fluorescent in situ hybridization, the method comprising:
(a) providing a sample comprising said cell;
(b) incubating the sample with a set of fluorescently labeled nucleic acid probes, wherein the set comprises one or more groups of probes, each group comprising first and second probes, each group bound to a unique combination of two or more fluorescent labels, said combination representing a single taxonomic or functional classification of cell; wherein the first probe of a group is bound to a first fluorescent label, and the second probe of the group is bound to a second fluorescent label; and wherein each probe of a group hybridizes to an identifier sequence present in a cell of the same unique taxonomic or functional classification;
(c) imaging the sample using a fluorescence microscope; and
(d) analyzing the image to identify said taxonomic or functional classification of said cell, wherein the combined presence in a cell of all of the fluorescent labels of a group identifies the cell as belonging to the taxonomic or functional classification represented by that group.

2. The method of claim 1, wherein the identifier sequence is a 16S ribosomal RNA or a 23S ribosomal RNA sequence.

3-4. (canceled)

5. The method of claim 1, wherein a functional classification is identified, and the functional classification comprises possessing a gene conferring resistance to an antibiotic or possessing a gene affecting metabolism.

6-9. (canceled)

10. The method of claim 1, wherein the sample comprises a mixed population of microbes, and a plurality of different taxonomic or functional classifications are identified.

11. The method of claim 10, wherein the sample comprises microbes that are functionally or metabolically linked.

12. The method of claim 10, further comprising the step of:
(e) determining a taxonomic classification distribution for the population.

13. The method of claim 12, further comprising repeating steps (a) through (e) after a time interval, and:
(f) comparing the taxonomic classification distributions to identify a change in said distribution over said time interval.

14. The method of claim 12, further comprising repeating steps (a) through (e) after contacting the population of microbes with a chemical, physical, or biological agent, and:
(f) comparing the taxonomic classification distributions to identify a change in said distribution in response to said agent.

15-18. (canceled)

19. The method of claim 14, wherein the agent is a biological agent selected from the group consisting of one or more viruses, one or more microbes, one or more eukaryotic cells, and a vaccine.

20-21. (canceled)

22. The method of claim 1, wherein the first and second probes of at least one group hybridize to the same identifier sequence.

23. The method of claim 1, wherein the first and second probes of at least one group hybridize to different identifier sequences.

24. The method of claim 1, wherein at least one group of probes further comprises a third probe, said third probe is bound to a third fluorescent label, and said first, second, and third probes form a unique combination of labels representing a single taxonomic or functional classification of cell.

25-31. (canceled)

32. The method of claim 1, wherein the set of fluorescently labeled oligonucleotides comprises n distinct fluorescent labels and not more than n(n−1)/2 groups of probes.

33-37. (canceled)

38. A set of fluorescently labeled nucleic acid probes comprising one or more groups of probes, each group comprising a first and a second probe; wherein the first probe of a group is bound to a first fluorescent label, and the second probe of a group is bound to a second fluorescent label; wherein the first and second fluorescent labels of each group of probes form a unique combination within the set; and wherein each probe of a group hybridizes to an identifier sequence present in cells of a unique taxonomic or functional classification.

39. The set of probes according to claim 38, comprising a probe that hybridizes to a 16S ribosomal RNA or a 23S ribosomal RNA.

40-43. (canceled)

44. The set of probes according to claim 38, wherein the probes are 15-30 bases in length.

45. (canceled)

46. The set of probes of claim 38, wherein the first and second probes of at least one group hybridize to the same identifier sequence.

47. The set of probes of claim 38, wherein the first and second probes of at least one group hybridize to different identifier sequences.

48. The set of probes of claim 38, wherein at least one group of probes further comprises a third probe, said third probe is bound to a third fluorescent label, and said first, second, and third labels form a unique combination within the set.

49-50. (canceled)

51. The set of probes of claim 38, wherein the set comprises n distinct fluorescent labels and not more than n(n−1)/2 groups of probes.

52-56. (canceled)

57. A kit comprising one or more sets of fluorescently labeled oligonucleotides according to claim 39 together with packaging materials and instructions for use.
